(19) United States
(12) Patent Application Publication
(10) Pub. No.: US 2008/0169290 A1
(43) Pub. Date: Jul. 17, 2008

(54) In-Wall Waste Receptacles For Hospital and Laboratory Environments

(76) Inventor: John R. Mangiardi, Greenwich, CT (US)

Correspondence Address: HUNTON & WILLIAMS/NEW YORK;INTELLECTUAL PROPERTY DEPT., 1900 K STREET, N.W., SUITE 1200, WASHINGTON, DC, 20006-1109, US

(21) Appl. No.: 11/996,026

(22) PCT Filed: Jul. 20, 2006
(86) PCT No.: PCT/US2006/028234

§ 371 Date: Feb. 6, 2008

Related U.S. Application Data
(60) Provisional application No. 60/701,106, filed on Jul. 20, 2005.

(51) Int. Cl. B65F 1/06; B65F 1/14

(52) U.S. Cl. 220/477; 220/495.11

(57) ABSTRACT

A trash receptacle comprising an entrance in a wall adapted to receive a disposable, lightweight, and preferably plastic cartridge is disclosed. The cartridge is adapted to dispose its center of gravity in a manner that maintains the cartridge within the wall entrance under its own empty weight. The cartridge may further receive a disposable bag to maintain sterility. The bag can be held in place by a sealing device, such as an O-ring, which, in one embodiment, snaps over said bag onto said cartridge, said O-ring or other sealing device preferably color-coded to indicate the type of waste to be disposed in said cartridge. The cartridge is also adapted to receive a plug to allow disposal of sharps. If a bag breaks, the cartridge may be sealed and disposed of, thereby maintaining a sanitary and sterile environment. The receptacles may be arrayed in a wall, thereby providing a convenient waste disposal system that does not interfere with critical working space.

### CROSS-REFERENCE TO RELATED APPLICATIONS

This application claims the benefit of provisional patent application U.S. Ser. No. 60/701,106, filed Jul. 20, 2005 by the present inventor. The contents of U.S. Ser. No. 60/701,106 are expressly incorporated herein by reference thereto.

The following references are hereby explicitly incorporated by reference thereto:
U.S. Pat. No. 4,925,048
Applications filed along with present application by current inventor on this date entitled:
   IN-CEILING FOCUS LOCATED SURGICAL LIGHTING
   HOSPITAL OPERATING ROOM RE-DESIGN
   AMBIENT LIGHTING IN HOSPITAL SURGICAL ENVIRONMENTS
   USE OF ULTRAVIOLET GERMICIDAL IRRADIATION IN HEALTH CARE ENVIRONMENTS
   MULTIFUNCTIONAL FLOOR PODS
   RE-DESIGN OF OPERATING ROOM TABLES
   ROBOTIC FLOOR CLEANING WITH STERILE, DISPOSABLE CARTRIDGES

### BACKGROUND OF THE INVENTION

1. Field of Invention

The present invention relates to a device and method for disposing of waste in medical or laboratory environments.

2. Background of the Invention

When waste is generated in the medical or scientific environment, it consumes valuable space. Typically, waste generated during a medical procedure or a scientific experiment is disposed in specially marked waste receptacles that exist as stand-alone containers such as the foot-maneuvered bucket of Noack in U.S. Pat. No. 4,925,048. For each type of waste, a different receptacle is usually required such as for sharps, regular trash, and biologics. An invention that can provide a disposal method that does not eliminate valuable space in the working environment would be of benefit. Further, medical or scientific environments typically require strictly sterile working conditions. A device that can incorporate a means for maintaining sterility in its design would be of further benefit.

It is therefore an object of this invention to provide a sterile, disposable receptacle that does not eliminate space in the working environment.

It is another object of this invention to provide a receptacle that is lightweight and cost-effective.

It is yet another object of this invention to integrate said receptacle with existing disposal means such as hospital biologic waste bags.

At least one of the above objects is met in whole or in part by the invention and additional objects are shown by the following description and claims.

### SUMMARY OF THE INVENTION

A trash receptacle is described. It comprises a molded, lightweight, plastic tube, closed at its bottom end, and adapted to fit within an opening in a wall. The tube has an opening that can receive a bag, said bag capable of being held in place by the tube after insertion of the tube into a specially adapted opening in the wall, said opening in the wall also comprised as part of the present invention. The wall is specially adapted to be capable of holding said tube by the weight of said tube alone. The tube is shaped such that after insertion of the tube into the opening in the wall, the center of gravity of the tube is adjacent to, as opposed to comprised somewhere within the tube, thereby helping maintain the tube stably within the opening in the wall. The tube is further adapted to allow a variety of plugs to fit within the opening of the tube to allow closure of the receptacle should disposal of the tube be necessary.

In a typical use, the empty tube has a bag inserted into the opening within the tube; the upper portion of the bag may be wrapped around the opening of the tube to help secure the bag. An O-ring or other sealing device, adapted to snap over the waste bag and onto the cartridge, may then be secured over the bag and cartridge. The O-ring or other sealing device is preferably color-coded and can snap over the bag and cartridge in a variety of manners. In one manner, the O-ring has a flexible hook which secures over the lip of the cartridge, or, alternatively, the O-ring may be fashioned like a standard kitchen container lid (like those manufactured by Tupperware®). In any embodiment of the O-ring, the cartridge is likewise adapted to secure to the O-ring. Other sealing devices, similar to O-rings but not necessarily shaped as an “O”, may also be used, such as a square sealing device.

The tube is then inserted into the wall opening. After the bag is full, the bag may be removed and a new bag inserted. The bag maintains the sterility of the cartridge and hence helps maintain the sterility of the environment. If a bag breaks, the tube may be closed with a plug and disposed of. Since the tube is made of lightweight plastic, disposal is easy and cost-conscious. The receptacles may also be fitted with special sharps plugs to allow disposal of sharp instruments such as needles or scapula tips. The special sharp plugs are adapted to prevent a person from being pricked or cut by the contents of the cartridge. In a preferred embodiment, the sharps plug snaps into the tube and after the tube is full, the sharps plug may be sealed by a plastic sheet that slides over the plugs opening and snaps permanently into place. Lastly, the wall openings may be arrayed on the wall to provide convenient access to a variety of disposal tubes. It is preferred that the wall openings, the O-ring snaps or other snapable securing devices, or all be color-coded for easy identification of the type of waste receptacle provided by the wall opening and tube combination.

### BRIEF DESCRIPTION OF THE DRAWINGS

The present invention can best be understood in connection with the accompanying drawings. It is noted that the invention is not limited to the precise embodiments shown in drawings, in which:

FIG. 1 is a perspective view of the tube showing the opening and one possible shape of the tube.

FIG. 2 is a perspective view of one possible embodiment of a sharps plug.

FIG. 3 is a perspective view of one possible embodiment of a waste tube plug.

FIG. 4 is a side-view detail of the tube being inserted into the wall with a waste bag inserted into the tube.

FIG. 5 is a direct onward view of one possible array of waste receptacles including a bottom sharps receptacle.

FIG. 6 is a side-view detail of the tube being inserted into the wall with a waste bag inserted into the tube and with the waste bag held into place by a snapable securing device.

### BRIEF DESCRIPTION OF REFERENCE NUMERALS

100 Tube Member Opening; 102 Tube Member Lip; 104 Tube Member Molded Surface; 106 Tube Member/Cartridge; 120 Sharps Closing Sheet; 122 Sharps Plug Opening; 124 Sharps Plug Surface; 126 Sharps Plug Protective House; 128 Sharps Plug; 130 Waste Plug; 132 Waste Plug Knob; 150 Wall Adapted For Tube Member; 154 Center Of Gravity Of Tube Member; 156 Waste Bag; 160 Wall Chamfer; 170 Trash Receptacle Array; 172 Trash Receptacles; 174 Biologics Receptacle; 176 Sharps Receptacle; 178 Color Coding Means; 200 Snapable Sealer; 202 Sealer Perimeter; 204 Sealer Snap

### DETAILED DESCRIPTION OF THE INVENTION

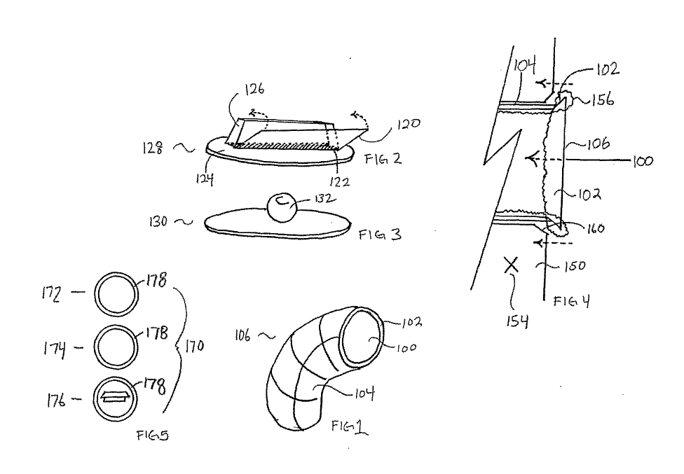

As seen in FIG. 1, the waste receptacle cartridge 106 has a preferred shape similar to a rounded upside-down “L”. Other shapes may used, provided they help hold the cartridge into wall. For example, a non-upside-down “L” shape would not be appropriate.

The outside molded surface can be a variety of textures but is preferably composed, as is the entirety of the cartridge 106, out of lightweight plastic. The shaping and texture may be adapted, as it is in the preferred embodiment, to allow a smooth sliding action into or out of the second wall-element. Alternatively, the texture may be adjusted to make movement more resistant.

The tube structure of 106 defines an interior 100. As can be seen, tube 106 has a bottom surface providing a tube with a single entrance. The outside perimeter of lip 102 is designed to fit snugly within the second wall-element and to allow a variety of plugs to fit inside the perimeter of the lip.

In FIG. 2, a sharps plug 128 is shown. The surface of the plug 124 terminates at the outside circumference that is adapted to fit inside the lip 102 of the cartridge/tube 106. At the center of the plug 124 is an opening 122 into the interior 100 of cartridge 106. The opening 122 is surrounded by house 126 that prevents accidental insertion of a user digit into the opening. Sheet 120 may close into the interior of house 126, thereby closing hole 122. House 126 is adapted to prevent 120 from reopening the hole at 122 by, for example, ridges adapted to allow one-way travel of 120 only (ridges not shown).

In FIG. 3, a typical embodiment of a cartridge plug 130 is shown. In a typical use, the interior waste bag 156 (not shown) inside cavity 100 of cartridge 106 is removed. A new, empty waste bag may then be inserted. If the bag is broken, plug 130 may be inserted snugly into the opening 100 and sealing around lip 102 using knob 132 as a handle.

FIG. 4 displays a side-view of the cartridge 106 and wall opening 150 combination with cartridge 106 and its surface 104 nearly fully inserted into the wall-opening. Cartridge 106 has a waste bag 156 inserted into cavity 100 and wrapped around the lip 102. As the cartridge is fully inserted into the wall opening, bag 156 is pressed against the outside of lip 102 and the chamfer 160. The chamfer/lip design is one typical embodiment. The chamfer can instead be a recessed notch with the lip adapted to join with said notch. As can be seen by the mark “X”, the center of gravity in all preferred embodiments is disposed below the entrance to cavity 100 and near the wall. This helps cartridge 106 maintain its position inside the wall cavity of 150 under its own weight.

FIG. 5 displays a possible arrangement of an array of waste receptacles having a rounded entrance. For example, waste receptacles 172, 174, and 176 may be trash, biologics, and sharps respectively, and may be further have color-coding means 178, such as by a colored sealing snap, a color lip perimeter, or a colored cartridge, thereby forming trash array 170. Color-coding works to identify the type of waste to be deposited in a cartridge; for example, a red sealing snap such as a red O-ring could be used to identify a cartridge as accepting biological waste, whereas a blue colored sealing snap could indicate regular trash waste.

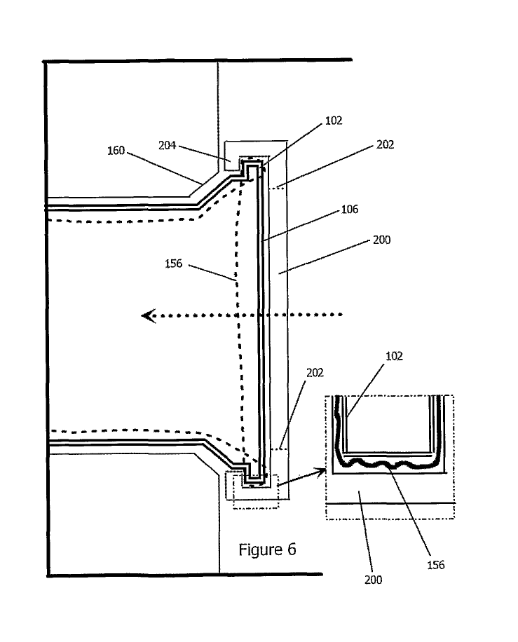

FIG. 6 displays an additional embodiment of the invention. With respect to the drawing, tube 106 is seen with bag 156 inserted, and tube and bag inserted into the wall opening resting against 160. The lip 102 in this embodiment is adapted to allow sealing device 200 to snap onto the lip 102 by snap 204. Alternatively, it is conceived that the sealing device will snap onto lip 102 as would a standard kitchen container lid such as those manufactured by Tupperware®. The above sharps and seal plugs may also be adapted with snaps to be held into place about the cartridge. Lines 202 demark the inner perimeter of the sealing device, which is (inside of the lines) open to allow access to the cartridge. Sealing device 200 functions to securely hold bag 156. The sealing devices are preferably color-coded to allow identification of the type of waste to be disposed of in the attached waste bag and or cartridge.

In the foregoing description, certain terms and visual depictions are used to illustrate the preferred embodiment. However, no unnecessary limitations are to be construed by the terms used or illustrations depicted, beyond what is shown in the prior art, since the terms and illustrations are exemplary only, and are not meant to limit the scope of the present invention. It is further known that other modifications may be made to the present invention, without departing the scope of the invention, as noted in the appended claims.

I claim:

1. A trash receptacle for hospital and laboratory environments, comprising
a cartridge which is adapted to be received and inserted into an opening in a wall, said wall in a hospital or laboratory environment,
said cartridge having a cavity and an opening to said cavity,
said cartridge adapted to seal against said wall,
said cartridge having a center of gravity not comprised on or within said cartridge, and
said cartridge capable of removal after insertion into said opening.

2. The trash receptacle of claim 1 further comprising a waste bag inside of said cavity, an upper portion of said waste bag optionally wrapped around the perimeter to said opening.

3. The trash receptacle of claim 1 further comprising a plug adapted to seal said opening.

4. The trash receptacle of claim 3 in which said plug is further adapted with a grip comprised on an outer surface of said plug.

5. The trash receptacle of claim 3 in which said plug is further adapted to provide a channel from a top of said plug to a bottom of said plug.

6. The trash receptacle of claim 5 in which said channel in said plug is surrounded by a protective house adapted to prevent accidental insertion of a user's digit and in which said channel in said plug may be sealed by a sheet that permanently shuts said channel.

7. The trash receptacle of claim 3 in which said plug is color-coded.

8. The trash receptacle of claim 1 in which the perimeter to said opening is color-coded to indicate the type of waste to be inserted into said cartridge's cavity.

9. The trash receptacle of claim 1 in which the perimeter to said opening comprises a recessed notch adapted to allow a user digit to be inserted thereby allowing said user digit to remove said cartridge.

10. The trash receptacle of claim 1 further comprising a sealing device, which snaps over a waste bag and onto a lip of said cartridge, said lip on the perimeter of said opening.

11. The trash receptacle of claim 10 in which said sealing device is color-coded.

12. The trash receptacle of claim 11 in which said sealing device is an O-ring.

13. A method of sanitary disposal of hospital or laboratory waste comprising
providing a trash receptacle as described in claim 11;
inserting a waste bag into said cartridge;
inserting said cartridge into said opening;
disposing of waste into said cartridge by placing said waste into said waste bag inside said cartridge;
sealing said cartridge with a plug adapted to seal said cartridge entrance if said waste bag is broken, removing said cartridge from said opening, inserting a new waste bag into a new cartridge, and then inserting said new cartridge into said opening;
removing said waste bag if full and unbroken and replacing said waste bag with a new waste bag.

14. The method of claim 13 further comprising the step of placing said color-coded sealing device over said waste bag after said waste bag is inserted into said cartridge and then snapping said sealing device onto said cartridge and also comprising the step of removing said color-coded sealing device before sealing of said cartridge or before removing of said waste bag.

15. A method of sanitary disposal of hospital or laboratory waste comprising
providing a trash receptacle as described in claim 6 in which said plug is color-coded;
inserting said cartridge into said opening;
disposing of waste into said cartridge by placing said waste through said channel in said plug into said cartridge cavity;
sealing said channel with said sheet when said cartridge cavity is full;
removing said cartridge from said opening;
replacing said cartridge and plug combination with a new cartridge and plug combination.
